(19) United States
(12) Patent Application Publication
(10) Pub. No.: US 2018/0265480 A1
(43) Pub. Date: Sep. 20, 2018

(54) METHOD FOR PREPARING CONTRAST AGENT PRECURSOR

(71) Applicant: INSTITUTE OF NUCLEAR ENERGY RESEARCH, ATOMIC ENERGY COUNCIL EXECUTIVE YUAN, R.O.C, TAOYUAN CITY (TW)

(72) Inventors: JHE-YU LIU, TAOYUAN CITY (TW); YU CHANG, TAOYUAN CITY (TW); CHENG-FANG HSU, TAOYUAN CITY (TW)

(21) Appl. No.: 15/459,468

(22) Filed: Mar. 15, 2017

(51) Int. Cl. C07D 255/02; A61K 49/00

(52) U.S. Cl. CPC C07D 255/02; A61K 49/0052

(57) ABSTRACT

A method for preparing a contrast agent precursor is revealed. The method includes several steps. First dissolve di-tert-butyl 2,2′-(1,4,7-triazacyclononane-1,4-diyl)diacetate, benzyl-2-bromoacetate and an alkaline compound in a first solvent to perform a bimolecular nucleophilic substitution reaction and get a product. Then dissolve the product in a second solvent to get a solution and adjust the pH value of the solution. Next add an organic solvent when the solution becomes acid and then remove a first organic layer that contains the organic solvent. Adjust the solution to become an alkaline solution and add the organic solvent again to get a second organic layer containing the organic solvent. The second organic layer contains the final product, di-tert-butyl 2,2′-{7-[2-(benzyloxy)-2-oxyethyl-(1,4,7-triazacyclononane-1,4-diyl}-diacetate.

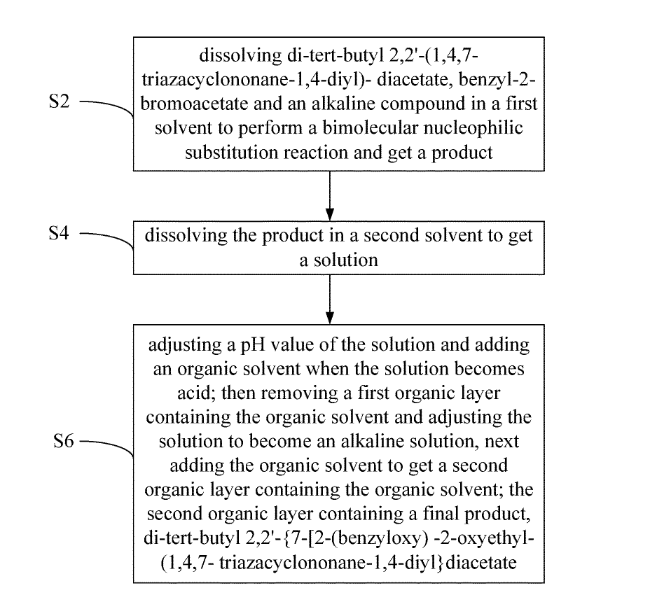

### BACKGROUND OF THE INVENTION

### Field of the Invention

The present invention relates to a method for preparing a contrast agent precursor, especially to a method for preparing a contrast agent precursor-2,2′-{7-[2-(benzyloxy)-2-oxyethyl-(1,4,7-triazacyclononane-1,4-diyl}diacetate.

Molecular imaging is a technology that allows users to observe in vivo changes and molecular transformation of tissues or cells in a non-invasive manner, including magnetic resonance imaging (MRI) and nuclear medicine imaging. The nuclear medicine imaging is further divided into two categories-positron emission tomography (PET) and single-photon emission computed tomography (SPECT).

Compared with conventional medical imaging technologies, molecular imaging features on its aid in early diagnosis. The conventional medical imaging is mainly used to observe final results of the changes in molecules. That means there are pathological changes of organs. In contrast, molecular imaging enables visualization of pathological processes in organs taking place at the cellular and molecular levels. As an emerging technology in the fields of nuclear medicine and molecular medicine, molecular imaging has become an important tool for diagnosis and research of dread diseases, especially malignant tumors.

Nuclear imaging uses low doses of radioisotopes linked to compounds that attach to specific tissues to be observed. The images of organs or tissues in living organisms are obtained by using special detection equipment that traces radioisotopes. Generally, radioisotopes are linked to contrast agent precursors and then used to prepare contrast agents for nuclear imaging. According to research results of Professor Eric W. Price in 2014, the contrast agent formed by 2,2′,2″-(1,4,7-triazacyclo-nonane-1,4,7-triyl)triacetic acid (abbreviated as NOTA) being chelated with Gallium-68 (⁶⁸Ga) is considered as having good radiolabeling efficiency and excellent in vivo stability.

For specific binding of the contrast agent to the tissues, the tissue specific peptide or protein is further bound to the contrast agent precursor. Thus the radioisotopes can be brought to the target tissue by recognition of peptide or protein.

However, the three carboxyl groups of NOTA are protected selectively by modification. Thus a lot of by-products are generated during bonding of NOTA to biomolecules such as peptides or proteins. Moreover, impurities are isolated by column chromatography in conventional preparation of NOTA. Thus not only preparation time is increased, the yield rate is decreased and the production cost is raised. Thus how to solve the problems of large amount of by-products and improve the preparation method has become an important issue.

### SUMMARY OF THE INVENTION

It is one object of the present invention to provide a method for preparing a contrast agent precursor in which di-tert-butyl 2,2′-(1,4,7-triazacyclononane-1,4-diyl)diacetate, benzyl-2-bromoacetate and an alkaline compound are dissolved in a solvent for performing a bimolecular nucleophilic substitution reaction to solve the problem of low yield rate caused by low solubility of the conventional catalyst in the solvent.

It is another object of the present invention to provide a method for preparing a contrast agent precursor that uses extraction to replace the conventional column chromatography for purification so as to reduce the preparation time dramatically and increase the yield rate. A product is dissolved to form a solvent and the solution is adjusted to acidic pH for extraction and removal of by-products. Then carry out extraction again to get a final product when the solution becomes alkaline.

In order to achieve the above objects, a method for preparing a contrast agent precursor according to the present invention includes the following steps. First dissolve di-tert-butyl 2,2′-(1,4,7-triazacyclononane-1,4-diyl)diacetate, benzyl-2-bromoacetate and an alkaline compound in a first solvent to perform a bimolecular nucleophilic substitution reaction and get a product. Then dissolve the product in a second solvent to get a solution and adjust the pH value of the solution and add an organic solvent when the solution becomes acid. Next remove a first organic layer that contains the organic solvent and adjust the solution to become an alkaline solution. Add the organic solvent to get a second organic layer containing the organic solvent. The second organic layer contains the final product, di-tert-butyl 2,2′-{7-[2-(benzyloxy)-2-oxyethyl-(1,4,7-triazacyclononane-1,4-diyl}diacetate.

Before the step of dissolving di-tert-butyl 2,2′-(1,4,7-triazacyclo-nonane-1,4-diyl)diacetate, benzyl-2-bromoacetate and an alkaline compound in a first solvent to perform a bimolecular nucleophilic substitution reaction, the di-tert-butyl 2,2′-(1,4,7-triazacyclononane-1,4-diyl)diacetate is dissolved in tetrahydrofuran or anhydrous acetonitrile.

The alkaline compound is triethylamine

The first solvent is tetrahydrofuran or anhydrous acetonitrile.

The reaction time of the bimolecular nucleophilic substitution reaction is ranging from 6 hours to 20 hours.

The bimolecular nucleophilic substitution reaction is carried out at room temperature.

The bimolecular nucleophilic substitution reaction is carried out to form a 2-benzyloxy group in di-tert-butyl 2,2′-(1,4,7-triazacyclononane-1,4-diyl)-diacetate.

The second solvent is water.

The organic solvent is ether or chloroform.

### BRIEF DESCRIPTION OF THE DRAWINGS

The structure and the technical means adopted by the present invention to achieve the above and other objects can be best understood by referring to the following detailed description of the preferred embodiments and the accompanying drawings, wherein:

The FIGURE is a flow chart showing steps of an embodiment according to the present invention.

### DETAILED DESCRIPTION OF THE PREFERRED EMBODIMENT

In order to learn features and functions of the present invention, please refer to the following embodiments and detailed descriptions.

A compound 2,2′,2″-(1,4,7-triazacyclononane-1,4,7-triyl)-triacetic acid (NOTA) is conventionally used for labeling radioisotopes. A lot of by-products are generated when NOTA is bound to biomolecules so that the products are complex. Moreover, the conventional technique purifies the products by column chromatography and the elution takes a long time. The products obtained by chromatography are amorphous oils so that no suitable solvent is available for recrystallization to get solid with fixed shape. Thus the present invention provides a novel method for preparing a compound in which different reactants are used to minimize impurities in products. The preparation process is simplified, the preparation time is effectively shortened and the production efficiency is improved.

The following are detailed description of the materials required, properties of the materials and the method for preparing the contrast agent precursor.

Refer to the FIGURE, a flow chart of a method for preparing a contrast agent precursor according to the present invention includes the following steps.

Step S2: Dissolve di-tert-butyl 2,2′-(1,4,7-triazacyclononane-1,4-diyl)diacetate, benzyl-2-bromoacetate and an alkaline compound in a first solvent to perform a bimolecular nucleophilic substitution reaction and get a product.

Step S4: Dissolve the product in a second solvent to get a solution.

Step S6: Adjust the pH value of the solution and add an organic solvent when the solution becomes acid. Then remove a first organic layer that contains the organic solvent and adjust the solution to become an alkaline solution. Add the organic solvent again to get a second organic layer containing the organic solvent. The second organic layer contains the final product, di-tert-butyl 2,2′-{7-[2-(benzyloxy)-2-oxyethyl-(1,4,7-triazacyclononane-1,4-diyl}diacetate.

As mentioned in the Step S2, di-tert-butyl 2,2′-(1,4,7-triazacyclononane-1,4-diyl)diacetate and benzyl-2-bromo-acetate are used as starting reactants dissolved together with an alkaline compound in a first solvent to carry out a bimolecular nucleophilic substitution reaction and get a product.

The alkaline compound used in the step S2 is a catalyst for the bimolecular nucleophilic substitution reaction. The alkaline compound should be compatible with the first solvent. That means the alkaline compound can be dissolved in the first solvent easily so as to prevent increasing complexity of the products obtained and reduction of the yield rate. In this embodiment, the catalyst is triethylamine and the solvent is either tetrahydrofuran or anhydrous acetonitrile. The catalyst and the solvent used can be, but not limited to, the compounds mentioned above.

As to the bimolecular nucleophilic substitution reaction performed in the above step, the reaction time is ranging from 6 hours to 20 hours and the reaction temperature is 20° C. to 30° C.

In the above bimolecular nucleophilic substitution reaction, benzyl-2-bromoacetate is used as a nucleophilic reagent. Lone-pair electrons on a bromide ion attack di-tert-butyl 2,2′-(1,4,7-triazacyclo-nonane-1,4-diyl)diacetate to form a 2-benzyloxy group.

In the step S4, the product obtained in the step S2 is concentrated and then the concentrated product is dissolved in a second solvent to get a solution. Then perform the following purification process. In this embodiment, the second solvent can be, but not limited to, water.

In the step S6, the solvent obtained in the step S4 is extracted and purified. In this embodiment, the purification is performed by acid-base extraction. Based on different acid-base properties, the impurities and the product are dissolved in the solvent and separated from each other in an acid or alkaline environment. Thus the products are purified.

First adjust the solution to acidic pH with hydrogen chloride and then add an organic solvent. At the moment, the impurities are dissolved in the organic solvent to form a first organic layer. Then remove the first organic layer containing the organic solvent and get an aqueous layer. Next adjust the aqueous layer to alkaline pH and then add an organic solvent. Now the product is dissolved in the organic solvent to form a second organic layer. Get the second organic layer, remove the organic solvent and dry the purified product. The extraction method mentioned above is the acid-base extraction. In this embodiment, the organic solvent can be, but not limited to, ether or chloroform.

The chemical reaction of the above preparation procedure is shown by the following equation one:

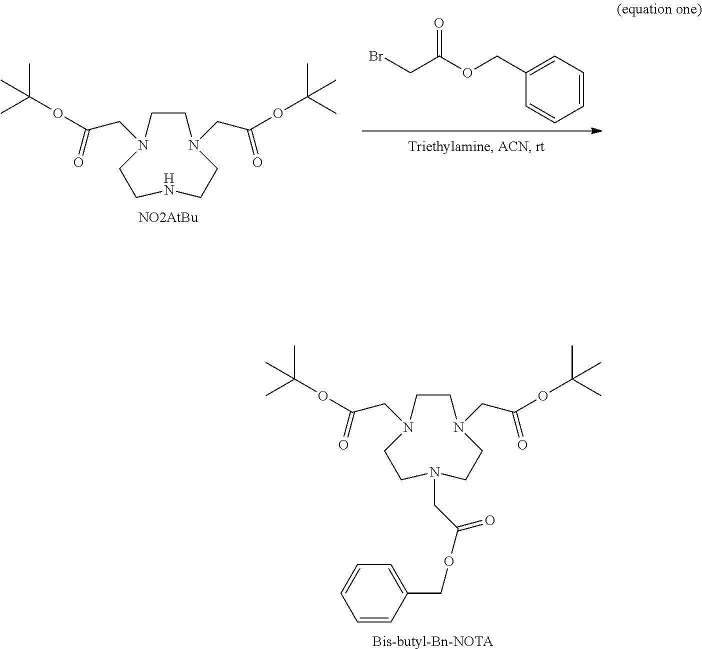

The contrast agent precursor prepared by the above procedure can further be linked with radioisotopes commonly employed in the nuclear medicine imaging such as technetium-99m (^{99m}Tc), iodine-123 (¹²³I), Gallium-68 (⁶⁸Ga), etc. In this embodiment, Gallium-68 is preferred. The pharmacokinetic properties of Gallium-68 are similar to those of most of peptides and small-molecular drugs. Gallium-68 also has good radiolabeling efficiency, excellent in vivo stability and higher blood clearance. Moreover, tissue-specific peptides or proteins are selected according to the target tissues intend to be detected and then bound to the contrast agent precursor of the present invention. Thus the peptides/proteins can recognize the tissue specifically and bring the radioisotopes to the target tissues. For example, integrin αᵥβ₃ is a receptor associated with tumor angiogenesis and cancer metastasis. An Arg-Gly-Asp (RGD) peptide can specifically bind integrin αᵥβ₃. Thus the contrast agent precursor can be specifically connected to tumor tissues by being bound to RGD.

In order to learn technical content, features and implementation of the present invention, please refer to the following embodiment.

### Synthesis of 2,2′-{7-[2-(benzyloxy)-2-oxyethyl-(1,4,7-triazacyclononane-1,4-diyl}diacetate

Dissolve the starting material-2.92 g, 8.17 mmol di-tert-butyl 2,2′-(1,4,7-triazacyclononane-1,4-diyl)diacetate in 20 ml in 20 ml anhydrous acetonitrile and add 1.3 ml 8.99 mmol triethylamine to form a solution. Then slowly add 1.5 ml 8.99 mmol benzyl-2-bromoacetate into the solution and stir overnight at room temperature to get a starting solution.

Concentrate the starting solution and dissolve the concentrated product in 30 ml water to get a product solution. Then use 1 N (normality) hydrogen chloride (HCl) to adjust the pH of the product solution to 3. Extract twice with 60 ml of ether for removing impurities in the product solution. Next take the aqueous layer of the product solution and adjust the pH of the product solution to 8. Then extract twice with 60 ml of ether and get the organic layer. Dry the organic layer to get 2.97 g yellow-white sticky solid, 2,2′-{7-[2-(benzyloxy)-2-oxyethyl-(1,4,7-triazacyclononane-1,4-diyl}diacetate.

### Analysis data of 2,2′-{7-[2-(benzyloxy)-2-oxyethyl-(1,4,7-triazacyclononane-1,4-diyl}diacetate

¹H NMR (CDCl₃, 300 MHz): δ 7.35 (m, 5H, H₁₉, H₂₀, H₂₁, H₂₂, H₂₃), 5.13 (s, 2H, H₁₇), 3.58 (s, 2H, H₁₅), 3.48 (s, 4H, H₇, H₁₁), 2.9-3.1 (m, 12H, H₁, H₂, H₃, H₄, H₅, H₆), 1.45 (s, 18H, H₁₀, H₄);

ESI-MS: m/z=506.46 (M+H)⁺.

In summary, a method for preparing a contrast agent precursor of the present invention uses triethylamine as a catalyst for the bimolecular nucleophilic substitution reaction between di-tert-butyl 2,2′-(1,4,7-triazacyclononane-1,4-diyl)diacetate and benzyl-2-bromo-acetate. The by-products generated are reduced so that the complexity of the products obtained is decreased. Moreover, acid-base extraction is used to replace the conventional column chromatography for product purification. The preparation time is reduced significantly and the production efficiency is increased. The recrystallization problem occurred in the conventional product purification can also be solved. Thus this is a breakthrough in the preparation of 2,2′-{7-[2-(benzyloxy)-2-oxyethyl-(1,4,7-triazacyclononane-1,4-diyl}diacetate.

Additional advantages and modifications will readily occur to those skilled in the art. Therefore, the invention in its broader aspects is not limited to the specific details, and representative devices shown and described herein. Accordingly, various modifications may be made without departing from the spirit or scope of the general inventive concept as defined by the appended claims and their equivalents.

1. A method for preparing a contrast agent precursor comprising the steps of:
dissolving di-tert-butyl 2,2′-(1,4,7-triazacyclononane-1,4-diyl)-diacetate, benzyl-2-bromoacetate and an alkaline compound in anhydrous acetonitrile to perform a bimolecular nucleophilic substitution reaction and get a product;
dissolving the product in a second solvent to get a solution;
adjusting the solution to become an acid solution and adding an ether when the solution becomes acid; then removing a first organic layer containing the organic ether; and
adjusting the solution to become an alkaline solution and adding the ether to get a second organic layer containing the ether; the second organic layer containing a final product, di-tert-butyl 2,2′-{7-[2-(benzyloxy)-2-oxyethyl-(1,4,7-triazacyclononane)-1,4-diyl]}diacetate.

2. The method as claimed in claim 1, wherein the di-tert-butyl 2,2′-(1,4,7-triazacyclononane-1,4-diyl)-diacetate is dissolved in tetrahydrofuran or anhydrous acetonitrile before the step of dissolving di-tert-butyl 2,2′-(1,4,7-triazacyclononane-1,4-diyl)-diacetate, benzyl-2-bromoacetate and an alkaline compound in the first solvent to perform a bimolecular nucleophilic substitution reaction.

3. The method as claimed in claim 1, wherein the alkaline compound is triethylamine

4. (canceled)

5. The method as claimed in claim 1, wherein reaction time of the bimolecular nucleophilic substitution reaction is ranging from 6 hours to 20 hours.

6. The method as claimed in claim 1, wherein the bimolecular nucleophilic substitution reaction is carried out at room temperature.

7. The method as claimed in claim 1, wherein the bimolecular nucleophilic substitution reaction is carried out to form a 2-benzyloxy group in di-tert-butyl 2,2′-(1,4,7-triazacyclononane)-1,4-diyl)-diacetate.

8. The method as claimed in claim 1, wherein the second solvent is water.

9. The method as claimed in claim 1, wherein the organic solvent is ether or chloroform.
